(19) United States
(12) Patent Application Publication
(10) Pub. No.: US 2022/0008579 A1
(43) Pub. Date: Jan. 13, 2022

(54) SCALABLE DISINFECTION SYSTEM

(71) Applicant: HermTac LLC, Dallas, TX (US)

(72) Inventors: Kevin Patel, Garland, TX (US); Thun Sanadi, Irving, TX (US); Caitlin Rawlins, Clyde, NC (US); Steve Morin, Bloomington, MN (US); Christopher Fowler, Saint Peterburg, FL (US)

(73) Assignee: HermTac LLC, Dallas, TX (US)

(21) Appl. No.: 17/339,304

(22) Filed: Jun. 4, 2021

Related U.S. Application Data
(60) Provisional application No. 63/048,801, filed on Jul. 7, 2020.

(51) Int. Cl. A61L 2/10; A61L 2/20; A61L 2/26; A61L 2/04

(52) U.S. Cl. CPC A61L 2/10; A61L 2/202; A61L 2/26; A61L 2202/26; A61L 2202/122; A61L 2202/123; A61L 2/04

(57) ABSTRACT

A disinfection panel for disinfecting an article facing the disinfection panel is provided. The disinfection panel comprises an interior surface, an exterior surface, a disinfecting member disposed on the interior surface, a first side comprising a first coupling member and a second side comprising a second coupling member. The disinfecting member is configured to disinfect the article facing the disinfection panel. The disinfecting member is any one or more of an ultraviolet (UV) light emitter, an ionizing or non-ionizing radiator, a chemical sprayer, an ozone sprayer, or a heat emitter.

### TECHNICAL FIELD

This disclosure relates to scalable disinfection system.

### BACKGROUND

Conventional disinfection systems typically have predetermined sizes. The sizes of those systems are predetermined at the manufacturing stage of the systems and are not adjustable.

However, different usages of the systems may require different optimal sizes for the usages. For example, a disinfection system having the size capable of accommodating a hospital bed may not be ideal for disinfecting a small backpack.

Also, because manufacturing disinfection systems having different sizes require different manufacturing stages or equipments, those conventional disinfection systems are typically more expensive.

Furthermore, those conventional disinfection systems typically have shapes that are not ideal for deploying (e.g., packing and shipping) the systems.

### SUMMARY

Thus, there is a need for a scalable disinfection system that can be assembled and disassembled easily.

Accordingly, in one aspect, a disinfection system for disinfecting an article contained within the disinfection system is provided. The disinfection system comprises a plurality of panels including a first panel and a second panel. The first panel comprises a first interior surface and a first exterior surface, and the second panel comprises a second interior surface and a second exterior surface. The disinfection system further comprises a plurality of disinfecting members including a first disinfecting member and a second disinfecting member. The first disinfecting member is disposed on the first interior surface and the second disinfecting member is disposed on the second interior surface. The first panel includes a first side, and the second panel includes a second side, the first side comprises a first coupling member, the second side comprises a second coupling member, the first coupling member is coupled (e.g., fastened) to the second side, and the second coupling member is coupled (e.g., fastened) to the first side.

In another aspect, a disinfection panel for disinfecting an article facing the disinfection panel is provided. The disinfection panel comprises an interior surface, an exterior surface, a disinfecting member disposed on the interior surface, a first side comprising a first coupling member, and a second side comprising a second coupling member. The disinfecting member is configured to disinfect the article facing the disinfection panel, and the disinfecting member is any one or more of an ultraviolet (UV) light emitter, an ionizing or non-ionizing radiator, a chemical sprayer, an ozone sprayer, or a heat emitter.

In another aspect, a method of providing a disinfection system for disinfecting an article contained within the disinfection system is provided. The method comprises obtaining a stack of a plurality of panels including a first panel and a second panel. The first panel comprises a first exterior surface, a first interior surface, and a first disinfecting member disposed on the first interior surface, and the second panel comprises a second exterior surface, a second interior surface, and a second disinfecting member disposed on the second interior surface. The method further comprises retrieving the first panel and the second panel from the stack of the plurality of panels. The first panel includes a first side, the second panel includes a second side, the first side comprises a first coupling member, and the second side comprises a second coupling member. The method further comprises coupling the first coupling member of the first panel to the second side of the second panel and coupling the second coupling member of the second panel to the first side of the first panel.

Embodiments of this disclosure provide a disinfection system that is scalable and can be assembled and disassembled easily. Making the disinfection system scalable allows the system to be adaptively used to disinfect articles of various different sizes. By making the disinfection system to be assembled and disassembled easily, the system can be packed/unpacked and shipped easily for easy deployment of the system.

### BRIEF DESCRIPTION OF THE DRAWINGS

The accompanying drawings, which are incorporated herein and form part of the specification, illustrate various embodiments.

FIG. 1 shows a disinfection system according to an embodiment.

FIG. 2 shows a disinfection system according to an embodiment.

FIG. 3 shows an enlarged view of a portion a disinfection system according to an embodiment.

FIG. 4 shows an enlarged view of a portion a disinfection system according to an embodiment.

FIGS. 5 and 6 show an arrangement of panels for forming a disinfection system according to an embodiment.

FIG. 7 shows a disinfection system according to an embodiment.

FIG. 8 shows a disinfection system according to an embodiment.

FIG. 9 shows an example of coupling member according to an embodiment.

### DETAILED DESCRIPTION

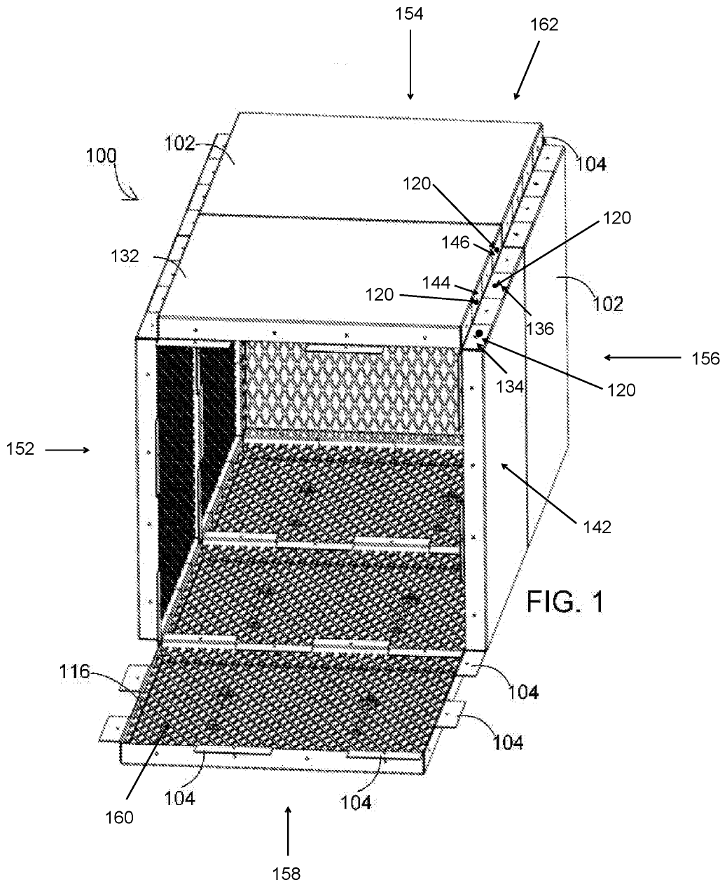

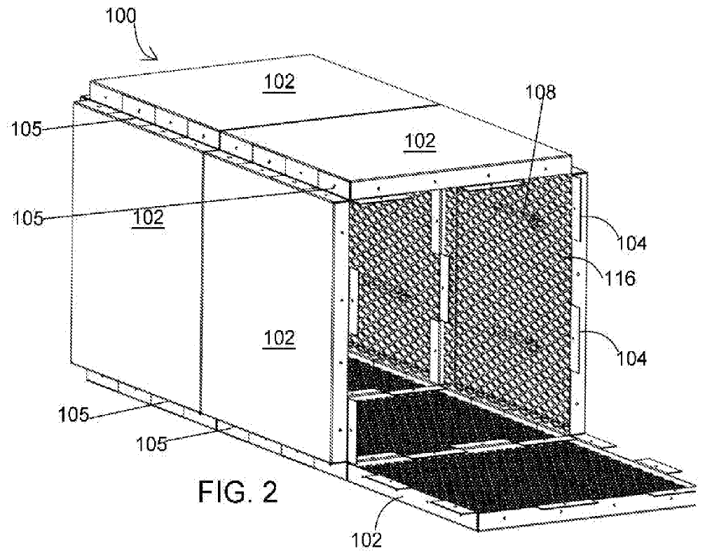

FIGS. 1 and 2 show a disinfection system (a.k.a, a sanitization system or a sterilization system) 100 according to some embodiments. The disinfection system 100 is configured to eliminate a substantial percentage of pathogens (e.g., viruses, bacteria, fungi, and mold spores) existing on articles (e.g., objects, animals, trees) located within the disinfection system 100.

In FIGS. 1 and 2, the disinfection system 100 has a shape of a rectangular box. However, the shape of the disinfection system 100 is not limited to a rectangular box. The disinfection system 100 may be shaped in any geometric structure having multiple surfaces. For example, in some embodiments, the disinfection system 100 may be shaped in the form of a hexagon box.

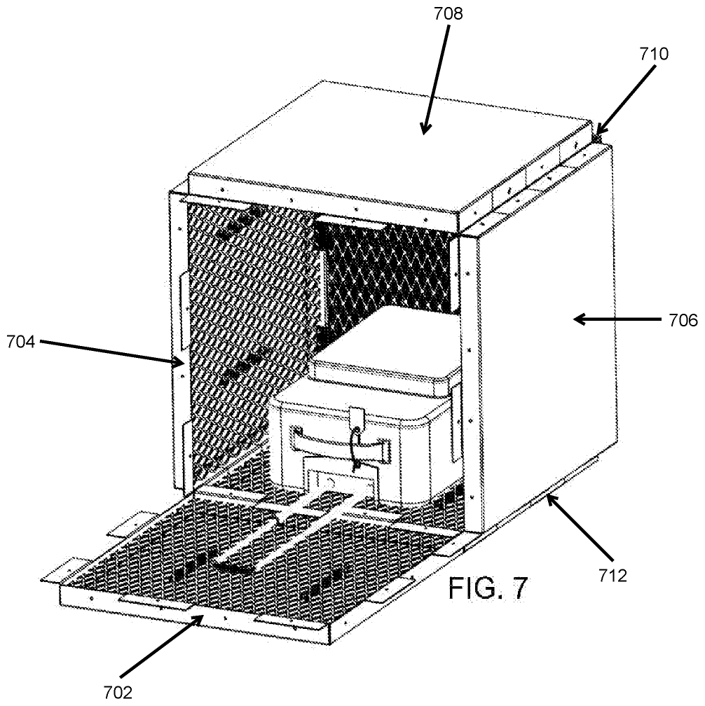

As shown in FIGS. 1 and 2, the disinfection system 100 may comprise two panels 102 on each of sides 152-158 and one panel on each of sides 160 and 162. The specific number of panels disposed on each side of the disinfection system 100 is provided in the figures of this disclosure for illustration purpose only and does not limit the embodiments of this disclosure in any way. For example, as shown in FIG. 7, the disinfection system 100 may comprise a single panel on each of the sides 152-162.

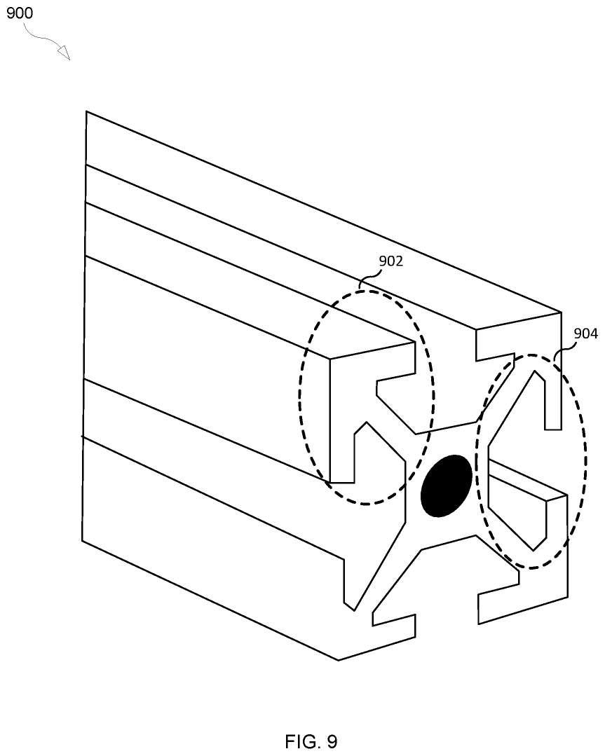

Referring back to FIGS. 1 and 2, each of the panels 102 may be coupled to at least one different panel 102 using an coupling member. The coupling members may be provided in different forms. For example, in FIGS. 1 and 2, the coupling members include flanges 104 and fasteners 105 (e.g., screws, bolts, cotter pins, etc.). In another example, framing extrusion 900 shown in FIG. 9 may be used as the coupling member. The extrusion 900 may comprise four protruding parts 902 and four openings 904. In one embodiment, one of the protruding parts 902 of the extrusion 900 included in one panel may be inserted into one of the openings 904 of the extrusion 900 included in another panel so that the two panels are attached to each other.

In FIGS. 1 and 2, the panels 102 include a top front panel 132 and a right front panel 142. The top front panel 132 comprises a first flange 134 and a second flange 136. The right front panel 142 comprises a first flange 144 and a second flange 146. In the disinfection system 100, the first flange 134 and the second flange 136 of the top front panel 132 are fastened to top surface 148 of the right front panel 142. Similarly, the first flange 144 and the second flange 146 of the right front panel 142 are fastened to side surface 138 of the top front panel 132.

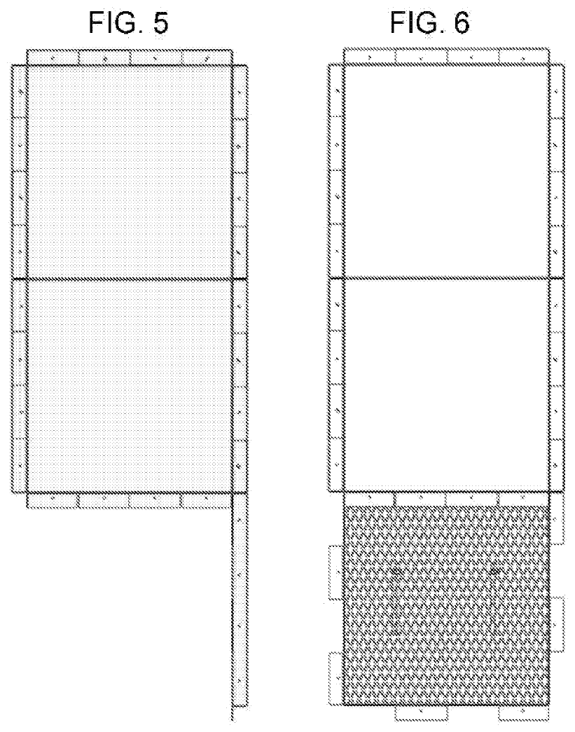

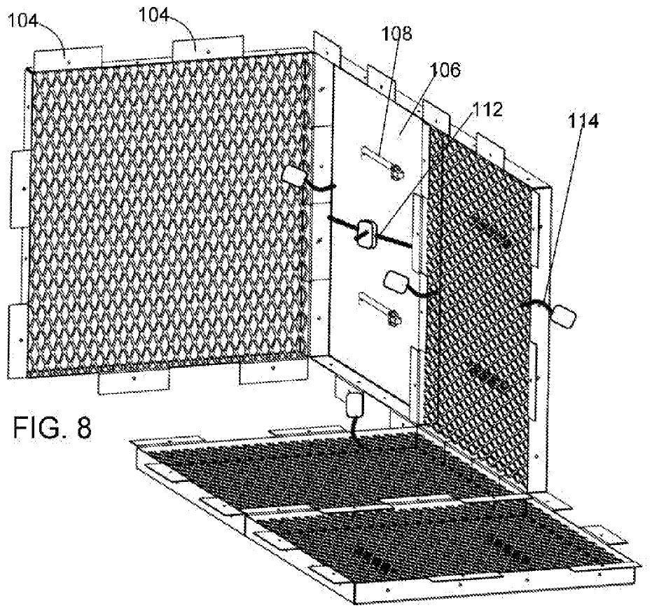

Each of the first flange 134 and the second flange 136 may comprise a hole 120. The first flange 134 and the second flange 136 may be fastened to the top surface 148 of the right front panel 142 by inserting the fastener 105 into the hole 120 included in each of the first flange 134 and the second flange 136. Similarly, each of the first flange 144 and the second flange 146 may comprise a hole 120. The first flange 144 and the second flange 146 may be fastened to the side surface 138 of the top front panel 132 by inserting the fastener 105 into the hole 120 included in each of the first flange 144 and the second flange 146. The specific number of flanges included in each panel shown in the figures of this disclosure is provided for illustration purpose only and does not limit the embodiments of this disclosure in any way. For example, even though FIG. 8 shows that each panel comprises two flanges on each side of each panel, in other embodiments, each panel may only comprise a single flange on each side of the panel. Alternatively, in other embodiments, each panel may comprise more than two flanges on each side of the panel. FIG. 5 shows a side view of the disinfection system 100 when the front panel 102 is opened. FIG. 6 shows a top view of the disinfection system 100 when the front panel 102 is opened.

As shown in FIGS. 1, 2, 5, and 6, in some embodiments, each of the two panels disposed on each of the sides 152-158 may not be connected to each other but may only be connected to the panels disposed on the other sides. For example, in FIG. 1, the front panel has an coupling member on each of the four sides of the front panel while each of the top panels has an coupling member on only two of the four sides of the top panel. In other words, each of the top panels does not have any coupling member connecting the top panel to another top panel.

As discussed above, the disinfection system according to the embodiments of this disclosure has a modular design. In other words, the disinfection system may comprise different modules—panels—that are assembled to form the system. Because of this design, the disinfection system according to the embodiments of this disclosure can be created to have various different sizes and shapes. Also while the figures of this disclosure shows that each panel is rectangular, any shape or size can be used for each panel in accordance with a tessellated configuration. For example, some alternative panels (not shown) may be disposed at the corners of large rectangular panels. Such alternative panels may have a special function or utility (e.g., including heating elements or infrared emitters).

In some embodiments, each panel 102 included in the disinfection system 100 may comprise one or more disinfecting members. The disinfecting members may include any one or more of an ultraviolet (UV) light emitter, an ionizing or non-ionizing radiator, a chemical sprayer (e.g., a means for misting), an ozone sprayer, or a heat emitter.

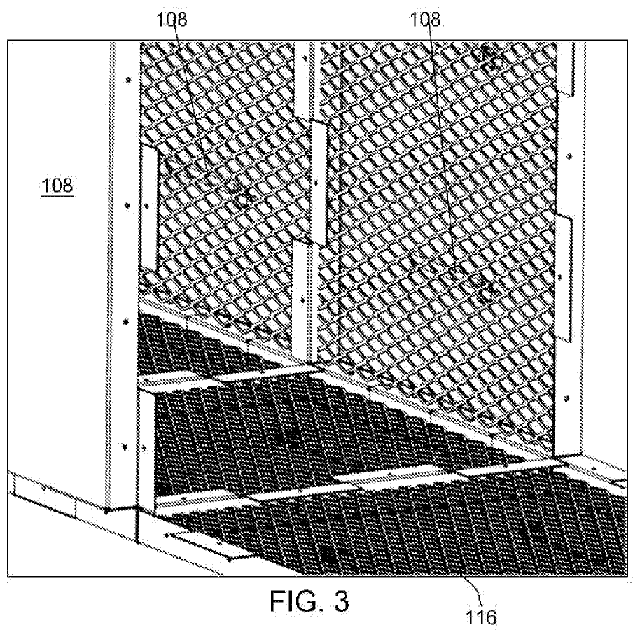

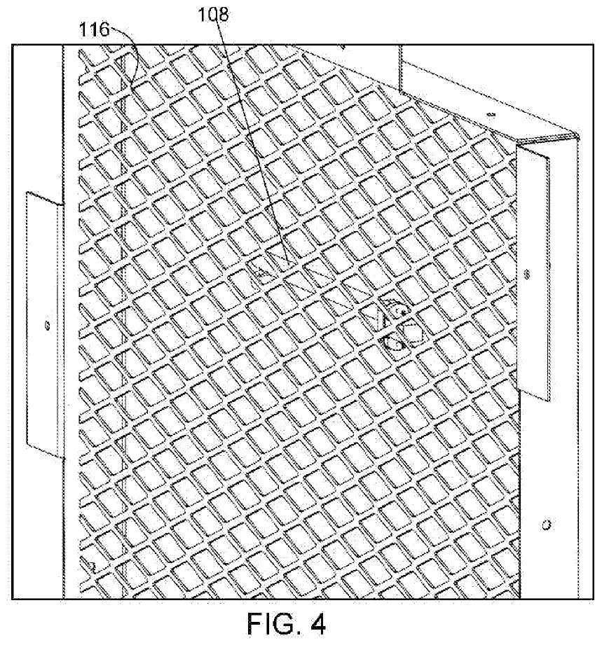

FIGS. 3 and 4 show the embodiments in which each of at least some panels 102 includes a UV light emitter 108 (e.g., a lower pressure mercury vapor lamp and/or a UVC light emitting diode (LED)). The UV light emitter 108 may be a UV-C light emitter or a far-UVC light emitter. The UV-C light emitter is capable of emitting UV light having a wavelength from 200 to 280 nanometers. This range of wavelength is capable of eliminating a substantial percentage of pathogens (e.g., viruses, bacteria, fungi, and mold spores). The far-UVC light emitter is capable of emitting UV light having a wavelength between 207 and 222 nanometers. Like UV-C light emitter, the wavelength of light that the far-UVC light emitter is capable of emitting UV light that is capable of eliminating a substantial percentage of pathogens (e.g., viruses, bacteria, fungi, and mold spores).

The UV light emitter 108 included in each of some panels 102 is configured to emit UV light toward the inside of the disinfection system 100 to disinfect an article (e.g., an object, an animal, a tree, a livestock) placed in the disinfection system 100. The article may be any object that needs to be disinfected. For example, in FIG. 7, the article is luggage 702.

Each panel 102 of the disinfection system 100 has an interior surface and an exterior surface. In some embodiments, a reflective material may be disposed on the interior surface of each panel 102. The reflective material may be polytetrafluoroethylene (PTFE) or aluminum (e.g., polished aluminum, reflective mylar, various PTFE films). The reflective material included in the interior surface of one panel is configured to reflect UV light emitted from the UV light emitter 108 included in other panel(s) such that the UV light is reflected toward the article disposed within the disinfection system 100.

Each panel 102 of the disinfection system 100 may comprise a power source configured to provide power to the disinfecting member included in each panel 102. For example, each panel 102 may include one or more batteries capable of providing power to the disinfecting member included in each panel. In some embodiments, the batteries may be rechargeable.

Alternatively, each panel 102 of the disinfection system 100 may comprise a power cord that can be inserted into a power outlet. In such embodiments, each panel 102 is independently powered.

In other embodiments, only one or a few of the panels 102 included in the system 100 comprises a power supply and each of the remaining panels comprises a power connector. For example, in the disinfection system 700 shown in FIG. 7, panel 702 may comprise a power supply and each of panels 704-712 may comprise a power connector (not shown). The power from the power supply included in the panel 702 may be delivered to the disinfecting member included in each of panels 704-712 via the power connector of each of panels 704-712. The power supply included in the panel 702 may be a battery or a power adapter that is configured to receive power from a power outlet. For example, the panel 702 may include a power cord that is connectable to an electric outlet. In some embodiments, the battery may be rechargeable.

In some embodiments, the power connector included in each panel may be a jumper wire capable of bridging adjacent panels. In other embodiments, the power connector included in each panel may include a power rail (not shown) and male/female electrical junctions along abutting surfaces of adjacent panels for the mating of the electrical connections.

In some embodiments, each of the disinfecting members may comprise a chemical sprayer or an ozone sprayer. In such embodiments, chemical(s) or material(s) needed for the chemical sprayer or the ozone sprayer need to be provided to each panel 102.

Thus, in some embodiments, each panel 102 may comprise a storage storing chemical(s) and/or material(s) that are needed for the operation of the chemical sprayer or the ozone sprayer. However, in other embodiments, each panel 102 comprises a tube for delivering the chemical(s) and/or the material(s) that are needed for the operation of the chemical sprayer or the ozone sprayer.

For example, referring back to FIG. 7, in the disinfection system 700 shown in FIG. 7, panel 702 may comprise a storage storing the chemical(s) and/or the material(s) that are needed for the operation of the chemical sprayer or the ozone sprayer and each of panels 704-712 may comprise a tube (not shown). The chemical(s) or the material(s) are delivered from the storage included in the panel 702 to the disinfecting member included in each of panels 704-712 via the tube included in each of panels 704-712. In other embodiments, the panel 702 may not include the storage but may include a tube receiving the chemical(s) or the material(s) from a storage located outside of the disinfection system 700. The panel 702 may forward the received chemical(s) or the component(s) to tubes of other panels.

FIG. 8 shows an example of tube 112. In FIG. 8, fluid, gas, or any other material needed for disinfecting operations may be sent (e.g., under pressure) to selected panels for disinfecting operation (e.g., chemical misting).

Referring back to FIGS. 3 and 4, as shown in the figures, the light emitter 108 is placed at the interior surface of each panel 102. Each panel 102 may further include a separator screen 116. The screen 116 may separate and protect (e.g., from accidental impact) the disinfecting member included in each panel (e.g., each light emitter 108) from the article placed in the system 100. In some embodiments, the screen 116 may comprise a plurality of holes through which the disinfecting means (e.g., UV light, heat, chemical gas, chemical mist, etc.) from the disinfecting member of each panel (e.g., the UV light emitter 108) travels. However, in other embodiments, the screen 116 may not comprise the holes. Instead, the screen 116 may be made of quartz or any material through which UVC light may pass through. Examples of such material include polymers (e.g., acrylic, silicone, etc.), high silica glasses (e.g., quartz, UV-grade fused silica, cheaper standard-grade fused silica, etc.), special UV glass compositions, borosilicate glass, some crystals/minerals (e.g., borate crystals like BBO, LBO, or artificial diamonds), florides (e.g., CaF2, MgF2, LiF, etc.), and polydimethylsiloxane (PDMS),

As shown in FIG. 7, in some embodiments, the disinfection system 100 is a closed system such that an article to be disinfected is fully contained within the disinfection system 100. However, in other embodiments, the disinfection system 100 is an open system. In other words, there may be one or more gaps between at least some of the panels included in the disinfection system 100. For example, in other embodiments, the disinfection system 100 may not comprise the panels 702 and 710 such that there are two openings in the system 100. In such case, a conveyor (e.g., a roller) may be installed on top of the interior surface of the panel 712. The conveyor is movable at least inside the system 100 such that an article to be disinfected can be disinfected by the system 100 by passing through the system 100 via the two openings. The conveyor may be motorized or may not be motorized. In some embodiments, the disinfection system 100 may have a collapsible geometric shape. For example, initially the disinfection system 100 may be in a collapsible state in which there is one or more gaps between different parts of the disinfection system 100. By applying force, the system 100 may be collapsible into a collapsed state in which the gaps disappear such that the system 100 fully seals the article located within the system 100.

The disinfection system 100 may be heat resistant and/or water resistant.

In some embodiments, each of one or more panels 102 may include a window via which disinfection progress can be monitored. Additionally or alternatively, each of one or more panels 102 may include a video camera that is coupled to a monitor disposed outside of the system 100. Using the camera and the monitor, disinfection progress can be monitored.

In some embodiments, each of one or more panels 102 may include a compartment in which additional disinfecting components can be added. The compartment may have a predetermined connection path needed for the disinfecting components and may be sealed after inserting the additional disinfecting component. For example, each panel 102 may include a compartment and one or more electrical connectors connected to the compartment. Thus, in case the panels 102 of the disinfection system 100 do not include a UV light emitter, a UV light emitter may be inserted into the compartment of one panel and one end of said one or more electrical connectors may be connected to the inserted UV light emitter and another end of the said one or more electrical connectors can be connected to an electrical connector included in other panel.

In some embodiments, each panel 102 may include a communicator. Each communicator may identify one or more functions of the panel. Additionally or alternatively, the communicator may indicate a location of the panel if the panel is used as a component of the disinfection system. For example, the communicator of a panel may indicate that the panel includes a UV light emitter or that the panel is to be used for a bottom side of the disinfection system. In a summary, a panel may detect one or more communicators of another panel and, based on the detected one or more communicators, identify any one or more of at least one component (e.g., a UV light emitter), at least one function (e.g., UV emission), or at least one predetermined location of said another panel (e.g., the bottom of the disinfection system).

Examples of the communicator include an RFID, an RFID reader, Bluetooth transceiver (or transmitter or receiver), or any communicator capable of communicating via radio access network (e.g., low-power RAN).

In some embodiments, a cart carrying at least partially assembled disinfection system may be provided. The cart may include a storage space in which the at least partially assembled disinfection system is stored. The storage space may be provided inside the cart or may be provided on top of the cart. The cart may include a means (e.g., a slider) for moving the disinfection system easily into or from the storage.

In a summary, according to some embodiments of this disclosure, in one aspect, a disinfection system for disinfecting an article contained within the disinfection system is provided. The disinfection system may comprise a plurality of panels including a first panel and a second panel. The first panel may comprise a first interior surface and a first exterior surface, and the second panel may comprise a second interior surface and a second exterior surface. The disinfection system may further comprise a plurality of disinfecting members including a first disinfecting member and a second disinfecting member. The first disinfecting member may be disposed on the first interior surface and the second disinfecting member may be disposed on the second interior surface. The first panel may include a first side, and the second panel may include a second side. The first side may comprise a first coupling member, the second side may comprise a second coupling member, the first coupling member may be fastened to the second side, and the second coupling member may be fastened to the first side.

In some embodiments, each of the plurality of disinfecting members is configured to disinfect the article contained within the disinfection system, and each of the plurality of disinfecting members is any one or more of an ultraviolet (UV) light emitter, an ionizing or non-ionizing radiator, a chemical sprayer, an ozone sprayer, or a heat emitter.

In some embodiments, the first coupling member comprises a first flange and a second flange, the second coupling member comprises a third flange and a fourth flange, the third flange is disposed between the first flange and the second flange in a particular view, and the second flange is disposed between the third flange and the fourth flange in a particular view.

In some embodiments, the disinfection system further comprises a plurality of separator screens including a first separator screen and a second separator screen. The first separator screen is disposed over the first interior surface and the first disinfecting member such that the first disinfecting member is disposed between the first separator screen and the first interior surface, and the second separator screen is disposed over the second interior surface and the second disinfecting member such that the second disinfecting member is disposed between the second separator screen and the second interior surface.

In some embodiments, the plurality of panels forms an enclosure surrounding all surface of the article contained within the disinfection system.

In some embodiments, the plurality of panels forms an enclosure with a first opening and a second opening, and the disinfection system further comprises a conveyor which is movable at least between the first opening and the second opening within the enclosure.

In some embodiments, the first disinfecting member comprises a first UV light emitter, the second disinfecting member comprises a second UV light emitter, and each of the first interior surface and the second interior surface comprises a reflective material for reflecting UV light.

In some embodiments, the reflective material is polytetrafluoroethylene (PTFE) or aluminum.

In some embodiments, the first disinfecting member comprises any one or more of a first chemical sprayer or a first ozone sprayer, the second disinfecting member comprises any one or more of a second chemical sprayer or a second ozone sprayer, the first panel comprises a first tube for carrying at least one disinfecting material needed for any one or more of the first chemical sprayer or the first ozone sprayer, the second panel comprises a second tube for carrying at least one disinfecting material needed for any one or more of the second chemical sprayer or the second ozone sprayer, and the first tube and the second tube are connected to each other.

In some embodiments, the first side of the first panel is the first interior surface and the second side of the second panel is the second interior surface.

In some embodiments, each of the panels includes a disinfecting member and a power source configured to provide power to the disinfecting member included in each of the panels.

In some embodiments, the first panel includes a power source configured to provide power to the first disinfecting member and a first power connector electrically connected to the power source, the second panel includes a second power connector electrically connected to the power source via the first power connector, and the power source is configured to provide power to the second disinfecting member via the second power connector and the first power connector.

In another aspect, a disinfection panel for disinfecting an article facing the disinfection panel is provided. The disinfection panel comprises an interior surface, an exterior surface, a disinfecting member disposed on the interior surface, a first side comprising a first coupling member, and a second side comprising a second coupling member. The disinfecting member is configured to disinfect the article facing the disinfection panel, and the disinfecting member is any one or more of an ultraviolet (UV) light emitter, an ionizing or non-ionizing radiator, a chemical sprayer, an ozone sprayer, or a heat emitter.

In some embodiments, the first coupling member comprises a first flange and a second flange, and the second coupling member comprises a third flange and a fourth flange.

In some embodiments, the disinfection panel further comprises a separator screen disposed over the interior surface and the disinfecting member such that the disinfecting member is disposed between the separator screen and the interior surface.

In some embodiments, the disinfecting member comprises a UV light emitter, and the interior surface comprises a reflective material for reflecting UV light.

In some embodiments, the reflective material is polytetrafluoroethylene (PTFE) or aluminum.

In some embodiments, the disinfecting member comprises any one or more of a chemical sprayer or an ozone sprayer, and the disinfection panel further comprises a tube for carrying at least one disinfecting material needed for any one or more of the chemical sprayer or the ozone sprayer.

In some embodiments, the disinfection panel further comprising a power source configured to provide power to the disinfecting member.

In some embodiments, the disinfection panel further comprising a power connector configured to be electrically connected to a power source that is located outside of the disinfection panel. The power source is configured to provide power to the disinfecting member via the power connector.

In another aspect, a method of providing a disinfection system for disinfecting an article contained within the disinfection system is provided. The method comprises obtaining a stack of a plurality of panels including a first panel and a second panel. The first panel comprises a first exterior surface, a first interior surface, and a first disinfecting member disposed on the first interior surface, and the second panel comprises a second exterior surface, a second interior surface, and a second disinfecting member disposed on the second interior surface. The method further comprises retrieving the first panel and the second panel from the stack of the plurality of panels. The first panel includes a first side, the second panel includes a second side, the first side comprises a first coupling member, and the second side comprises a second coupling member. The method further comprises coupling (e.g., fastening) the first coupling member of the first panel to the second side of the second panel, and coupling (e.g., fastening) the second coupling member of the second panel to the first side of the first panel.

In some embodiments, the first coupling member comprises a first flange and a second flange, and the second coupling member comprises a third flange and a fourth flange. Fastening the first coupling member to the second side comprises (i) putting a surface of the first flange to the second side and inserting a fixing member (e.g., a screw, a bolt, a pin, etc.) into a hole included in the second side via a hole included in the first flange and (ii) putting a surface of the second flange to the second side and inserting a fixing member (e.g., a screw, a bolt, a pin, etc.) into a hole included in the second side via a hole included in the second flange. Similarly, fastening the second coupling member to the first side comprises (i) putting a surface of the third flange to the first side and inserting a fixing member (e.g., a screw, a bolt, a pin, etc.) into a hole included in the first side via a hole included in the third flange and (ii) putting a surface of the fourth flange to the first side and inserting a fixing member (e.g., a screw, a bolt, a pin, etc.) into a hole included in the first side via a hole provided in the fourth flange.

In some embodiments, the method further comprises repeating the steps included in the method described above for other panels included in the stack of the panels such that the plurality of panels forms an enclosure surrounding all surface of the article contained within the disinfection system.

In some embodiments, the method further comprises repeating the steps included in the method described above for other panels included in the stack of the panels such that the plurality of panels forms an enclosure with a first opening and a second opening.

In some embodiments, the first disinfecting member comprises any one or more of a first chemical sprayer or a first ozone sprayer, the second disinfecting member comprises any one or more of a second chemical sprayer or a second ozone sprayer, the first panel comprises a first tube for carrying at least one disinfecting material needed for any one or more of the first chemical sprayer or the first ozone sprayer, and the second panel comprises a second tube for carrying at least one disinfecting material needed for any one or more of the second chemical sprayer or the second ozone sprayer. The method may further comprise connecting the first tube to the second tube.

In some embodiments, the first panel includes a power source configured to provide power to the first disinfecting member and a first power connector electrically connected to the power source, and the second panel includes a second power connector. The method may further comprise connecting the first power connector to the second power connector such that the power source is configured to provide power to the second disinfecting member via the second power connector and the first power connector.

As described above, the disinfection system according to the embodiments of this disclosure may be used to sanitize objects of different sizes (e.g., personal belongings such as a bag or a luggage, a hospital bed, a livestock, trees, fruits, animals, packages, food products, medical equipments, personal protective equipments (PPE), and animals), for example, prior to shipping, before/after delivery, and/or entry into a building or an airplane. Via the sanitization, viruses (such as COVID viruses), bacteria, fungi, and/or mold spores existing on the surfaces of the objects can be eliminated substantially.

As further described above, a disinfection system according to some embodiments of this disclosure has a modular design. In other words, the disinfection system may comprise a plurality of modules—panels—that are connected together. This modular design allows the disinfection system to be highly customizable and highly upgradable. Because different numbers of panels can be used in different ways to build a disinfection system based on desired needs, the disinfection system can be customized or upgraded to accommodate objects of different sizes and shapes. For example, four panels may be used to build a disinfection system having a long rectangular box shape (“I” shape) to disinfect a backpack or a small personal belonging while more than 20 panels may be used to build a disinfection system having a “T” shape designed to disinfect a tree.

Furthermore, this modular design allows a disinfection system to be built at a place where the disinfection system is needed or near the place where the disinfection system is needed. In other words, instead of shipping the entire disinfection system, the panels needed to build the disinfection system can be shipped and the disinfection system can be built using the panels at a desired location. Because the combined size of the panels is much smaller than the size of the entire disinfection system, the cost of the delivery associated with the disinfection system can be lowered substantially. Also, because each panel is in the form of a substantially flat plate, it is easier to pack (i.e., flat-packable) and ship the panels as compared to packing and shipping the entire disinfection system. For example, the sizes of some disinfection systems may be too large to go through some standard sized doors (e.g., ADA doors) but the size of each panel may be small enough to go through those doors.

Lastly, because the disinfection system is a modular system comprising a plurality of panels, and all or most of the panels needed for building a disinfection system are identical or substantially similar, the manufacturing cost of the disinfection system can be lowered substantially as compared to a conventional disinfection system that does not have a modular design. The manufacturing cost can further be lowered as compared to the conventional disinfection system because manufacturing each panel involves simple processes of cutting and folding a material.

While various embodiments are described herein, it should be understood that they have been presented by way of example only, and not limitation. Thus, the breadth and scope of this disclosure should not be limited by any of the above-described exemplary embodiments. Moreover, any combination of the above-described elements in all possible variations thereof is encompassed by the disclosure unless otherwise indicated herein or otherwise clearly contradicted by context.

Additionally, while the processes described above and illustrated in the drawings are shown as a sequence of steps, this was done solely for the sake of illustration. Accordingly, it is contemplated that some steps may be added, some steps may be omitted, the order of the steps may be re-arranged, and some steps may be performed in parallel.

Lastly, it is intended that this disclosure encompasses any further modifications, changes, rearrangements, substitutions, alternatives, design choices, and embodiments as would be appreciated by those of ordinary skill in the art having benefit of this disclosure and falling within the spirit and scope of the following claims.

1. A disinfection system for disinfecting an article contained within the disinfection system, the disinfection system comprising:
a plurality of panels including a first panel and a second panel, wherein the first panel comprises a first interior surface and a first exterior surface, and the second panel comprises a second interior surface and a second exterior surface; and
a plurality of disinfecting members including a first disinfecting member and a second disinfecting member, wherein the first disinfecting member is disposed on the first interior surface and the second disinfecting member is disposed on the second interior surface, further wherein
the first panel includes a first side, and the second panel includes a second side,
the first side comprises a first coupling member,
the second side comprises a second coupling member,
the first coupling member is coupled to the second side, and
the second coupling member is coupled to the first side.

2. The disinfection system of claim 1, wherein
each of the plurality of disinfecting members is configured to disinfect the article contained within the disinfection system, and
each of the plurality of disinfecting members is any one or more of an ultraviolet (UV) light emitter, an ionizing or non-ionizing radiator, a chemical sprayer, an ozone sprayer, or a heat emitter.

3. The disinfection system of claim 1, wherein
the first coupling member comprises a first flange and a second flange,
the second coupling member comprises a third flange and a fourth flange,
the third flange is disposed between the first flange and the second flange in a particular view, and
the second flange is disposed between the third flange and the fourth flange in a particular view.

4. The disinfection system of claim 1, the disinfection system further comprising:
a plurality of separator screens including a first separator screen and a second separator screen, wherein
the first separator screen is disposed over the first interior surface and the first disinfecting member such that the first disinfecting member is disposed between the first separator screen and the first interior surface, and
the second separator screen is disposed over the second interior surface and the second disinfecting member such that the second disinfecting member is disposed between the second separator screen and the second interior surface.

5. The disinfection system of claim 1, wherein the plurality of panels forms an enclosure surrounding all surface of the article contained within the disinfection system.

6. The disinfection system of claim 1, wherein
the plurality of panels forms an enclosure with a first opening and a second opening, and
the disinfection system further comprises a conveyor which is movable at least between the first opening and the second opening within the enclosure.

7. The disinfection system of claim 1, wherein
the first disinfecting member comprises a first UV light emitter,
the second disinfecting member comprises a second UV light emitter, and
each of the first interior surface and the second interior surface comprises a reflective material for reflecting UV light.

8. The disinfection system of claim 7, wherein the reflective material is polytetrafluoroethylene (PTFE) or aluminum.

9. The disinfection system of claim 1, wherein
the first disinfecting member comprises any one or more of a first chemical sprayer or a first ozone sprayer,
the second disinfecting member comprises any one or more of a second chemical sprayer or a second ozone sprayer,
the first panel comprises a first tube for carrying at least one disinfecting material needed for any one or more of the first chemical sprayer or the first ozone sprayer,
the second panel comprises a second tube for carrying at least one disinfecting material needed for any one or more of the second chemical sprayer or the second ozone sprayer, and
the first tube and the second tube are connected to each other.

10. The disinfection system of claim 1, wherein the first side of the first panel is the first interior surface and the second side of the second panel is the second interior surface.

11. The disinfection system of claim 1, wherein
the first panel includes a power source configured to provide power to the first disinfecting member and a first power connector electrically connected to the power source,
the second panel includes a second power connector electrically connected to the power source via the first power connector, and
the power source is configured to provide power to the second disinfecting member via the second power connector and the first power connector.

12. A disinfection panel for disinfecting an article facing the disinfection panel, the disinfection panel comprising:
an interior surface;
an exterior surface;
a disinfecting member disposed on the interior surface;
a first side comprising a first coupling member; and
a second side comprising a second coupling member, wherein
the disinfecting member is configured to disinfect the article facing the disinfection panel, and
the disinfecting member is any one or more of an ultraviolet (UV) light emitter, an ionizing or non-ionizing radiator, a chemical sprayer, an ozone sprayer, or a heat emitter.

13. The disinfection panel of claim 12, wherein
the first coupling member comprises a first flange and a second flange, and
the second coupling member comprises a third flange and a fourth flange.

14. The disinfection panel of claim 12, the disinfection panel further comprising:
a separator screen disposed over the interior surface and the disinfecting member such that the disinfecting member is disposed between the separator screen and the interior surface.

15. The disinfection panel of claim 12, wherein
the disinfecting member comprises a UV light emitter, and
the interior surface comprises a reflective material for reflecting UV light.

16. The disinfection panel of claim 15, wherein the reflective material is polytetrafluoroethylene (PTFE) or aluminum.

17. The disinfection panel of claim 12, wherein
the disinfecting member comprises any one or more of a chemical sprayer or an ozone sprayer, and
the disinfection panel further comprises a tube for carrying at least one disinfecting material needed for any one or more of the chemical sprayer or the ozone sprayer.

18. The disinfection panel of claim 12, the disinfection panel further comprising a power source configured to provide power to the disinfecting member.

19. The disinfection panel of claim 12, the disinfection panel further comprising a power connector configured to be electrically connected to a power source that is located outside of the disinfection panel, wherein
the power source is configured to provide power to the disinfecting member via the power connector.

20. A method of providing a disinfection system for disinfecting an article contained within the disinfection system, the method comprising:
obtaining a stack of a plurality of panels including a first panel and a second panel, wherein (i) the first panel comprises a first exterior surface, a first interior surface, and a first disinfecting member disposed on the first interior surface, and (ii) the second panel comprises a second exterior surface, a second interior surface, and a second disinfecting member disposed on the second interior surface;
retrieving the first panel and the second panel from the stack of the plurality of panels, wherein (i) the first panel includes a first side, (ii) the second panel includes a second side, (iii) the first side comprises a first coupling member, and (iv) the second side comprises a second coupling member, and
coupling the first coupling member of the first panel to the second side of the second panel; and
coupling the second coupling member of the second panel to the first side of the first panel.
